(19) United States
(12) Patent Application Publication
(10) Pub. No.: US 2017/0303953 A1
(43) Pub. Date: Oct. 26, 2017

(54) Surgical Instruments with Selectively Rotating Handles

(71) Applicants: Massachusetts Institute of Technology, Cambridge, MA (US); The Brigham and Women's Hospital, Inc., Boston, MA (US)

(72) Inventors: Adam Libert, Dover, MA (US); Jiayin Ling, Cambridge, MA (US); Daniel Jesus Gonzalez, Rutherford, NJ (US); Man-Chi Liu, Cambridge, MA (US); Nicholas Sazdanoff, Boston, MA (US); Joseph Ciccone, Chestnut Hill, MA (US); Joshua Kaplan, Philadephia, PA (US)

(21) Appl. No.: 15/509,230

(22) PCT Filed: Sep. 9, 2015
(86) PCT No.: PCT/US15/49180

§ 371 Date: Mar. 7, 2017

Related U.S. Application Data
(60) Provisional application No. 62/049,858, filed on Sep. 12, 2014.

(51) Int. Cl. A61B 17/3205; A61B 17/00

(52) U.S. Cl. CPC A61B 17/3205; A61B 17/00234; A61B 2017/00469

(57) ABSTRACT

This disclosure relates to surgical instruments with selectively rotating handles. In certain aspects, the surgical device includes a conduit having a longitudinal axis and configured to extend into a body lumen, and a handle coupled to the conduit and configured to selectively rotate relative to the conduit. The handle is coupled to the conduit by a connection including a clutch mechanism, and the handle is configured to rotate in response to a user activation of the clutch mechanism.

### CROSS-REFERENCE TO RELATED APPLICATIONS

This application claims the benefit of U.S. Provisional Patent Application No. 62/049,858, entitled “Surgical Instruments with Selectively Rotating Handles,” filed on Sep. 12, 2014. The disclosure of the foregoing application is incorporated herein by reference in its entirety for all purposes.

### STATEMENT AS TO FEDERALLY SPONSORED RESEARCH

This invention was made with Government support under grant No. W81XWH-09-2-0001 awarded under the U.S. Army Medical Research Acquisition Activity Cooperative Agreement. The Government has certain rights in the invention.

### TECHNICAL FIELD

This disclosure relates to surgical instruments with selectively rotating handles.

### BACKGROUND

More than 150,000 transurethral resection (“TUR”) procedures are performed in the U.S. each year to treat bladder cancer and/or an enlarged prostate. Commonly, a resectoscope is employed transurethrally to perform prostate and/or bladder surgery. This device has an elongated section provided with an outer sheath that is inserted into the urethra. The outer sheath prevents the urethra from contracting, while working elements within the outer sheath are employed to cut away the desired tissue. An inner sheath is connected to an irrigation system for washing away debris from the area. Commonly, a cutting element of the resectoscope is a conductive wire that is selectively heated through an electrical connection in the device. During use, the surgeon extends the cutting element beyond the end of the outer sheath to a position engaging the tissue to be cut. Thereafter, the cutting element is energized while the cutting element is manually retracted. As a result, tissue is separated from the targeted area. The surgeon views the affected area through a telescopic system that also is mounted within the outer sheath of the device, and repeatedly reorients the cutting element and repeats the cutting motion until the desired tissue removal is complete.

### SUMMARY

In general, this disclosure relates to surgical device assemblies for endoscopic surgeries and related components and methods including a handle that selectively and controllably rotates relative to the device and relative to a surgical tool arranged at a distal end of the device. The surgical device assemblies can be used, for example, for removing tissue during endoscopic procedures and/or endoscopic diagnostic procedures.

In one aspect, the disclosure features surgical devices that include a conduit having a longitudinal axis and being configured to extend into a body lumen, and a handle coupled to the conduit and configured to selectively rotate relative to the conduit. The handle is coupled to the conduit by a connection including a clutch mechanism, and the handle is configured to rotate in response to activation of the clutch mechanism, e.g., by a user.

In another aspect, the disclosure features resectoscopes that include a conduit, a surgical tool, an inner sheath, and an outer sheath. The conduit has a longitudinal axis and extends through an opening in the handle. The conduit is coupled to the conduit by a connection including a clutch mechanism. The handle is configured to selectively rotate relative to the conduit in response to activation of the clutch mechanism, e.g., by a user. The surgical tool is attached to the conduit and rotationally fixed relative to the conduit. The inner sheath is releasably attached to the conduit, and the inner sheath surrounds at least a portion of the conduit and the surgical tool. The outer sheath is releasably attached to the inner sheath, and the outer sheath surrounds at least a portion of the inner sheath.

In yet another aspect, the disclosure features uses of the surgical devices disclosed herein in methods that include first inserting a surgical device disclosed herein into a body cavity, rotating a conduit to position the surgical tool within the body cavity, and selectively rotating a handle. The surgical device includes a conduit having a longitudinal axis. The conduit extends through an opening in the handle. The handle is coupled to the conduit and a surgical tool is rotationally fixed to the conduit. The handle is configured to selectively rotate relative to the conduit, and the handle is coupled to the conduit by a connection including a clutch mechanism. The surgical tool is configured to reciprocate parallel to the surgical device, e.g., as in a resectoscope. The handle is selectively rotated about the conduit in response to a user activation of the clutch mechanism, wherein the handle rotates relative to the conduit and the surgical tool.

Various implementations of these devices and methods can include one or more of the following features.

In some implementations, the user activation includes retracting the handle. In certain implementations, the clutch mechanism includes one or more members aligned with one or more holes. In some implementations, the one or more members are fixed to the handle and the clutch mechanism can be configured to have an engaged state and a disengaged state, wherein the engaged state rotationally fixes the handle to the conduit.

In certain implementations, the surgical tool is attached to the conduit and aligned parallel to the longitudinal axis of the conduit. In some implementations, the surgical tool is rotationally fixed relative to the conduit. In certain implementations, the surgical tool is a cutting element, e.g., a cautery loop. In certain implementations, the surgical tool is configured to selectively extend from the outer sheath.

The new devices and methods provide various advantages. For example, providing a selectively rotating handle can permit a user, e.g., a surgeon, to maintain a comfortable grasp on the device throughout a resection procedure, including a resection procedure generally requiring the cutting element to be rotated a full 360 degrees, without having to overuse larger accessory muscles such as the deltoids. In addition, the new devices and methods allow the user to complete the procedure, e.g., a transurethral resection of a bladder tumor (“TURBT”) or a transurethral resection of the prostate (“TURP”), without requiring the user to release the handle, which facilitates a more efficient and effective procedure, benefitting both the user and the patient.

The rotation of the handle can also decrease the number of uncomfortable and imprecise hand movements through the rotation of the handle relative to the cutting element, such that the handle remains ergonomically oriented regardless of the rotation of the cutting element. This ergonomic position can also reduce the risk of injury to patients and can improve the overall quality of the procedure, because the user retains more control over the device throughout the procedure. In addition, the rotation of the handle is selectable such that the user can cause a handle rotation according to their preference while the functionality and configuration of the device are otherwise analogous to known devices.

The manner in which the clutching mechanism disconnects the surgical tool both physically and electrically with increased back force by the user, e.g., a surgeon, also imparts ancillary benefits. First, if the surgeon attempts to resect tissue that is too dense or dangerous to resect in this fashion (e.g., fibrotic tissue, the beak of the scope, stones, other foreign bodies), the loop will disengage, thereby preventing inadvertent and potentially serious tissue injury, as well as scope damage. Additionally, as the life of a surgical tool, such as a cautery loop, can be impacted by tension and bending, this would also help prevent damage to the surgical tool, e.g. a cautery loop, and extend its functional life.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and from the following claims.

### DESCRIPTION OF DRAWINGS

FIG. 1 is a side view of a disassembled resectoscope of the prior art.

FIG. 2 is a side view of a working element as described herein that includes a rotatable handle.

FIG. 3 is a perspective view of the working element of FIG. 2.

FIG. 4A is a partial cutaway view of a handle of a device described herein showing the internal components including a clutch mechanism of the working element of FIG. 2. The handle and clutch mechanism are shown in an engaged state.

FIG. 4B is a partial cutaway view of the handle of FIG. 4A showing the internal components including a clutch mechanism of the working element of FIG. 2. The handle and clutch mechanism are shown in a disengaged state.

FIG. 5 is an exploded view of the working element of FIG. 2.

FIG. 6A is rear perspective view the working element of FIG. 2.

FIG. 6B is a cutaway view of FIG. 6A.

FIGS. 7A-7D are a series of perspective views of the resectoscope as described herein during rotation.

FIGS. 8A-8C are views of alternative clutch engagement mechanisms.

### DETAILED DESCRIPTION

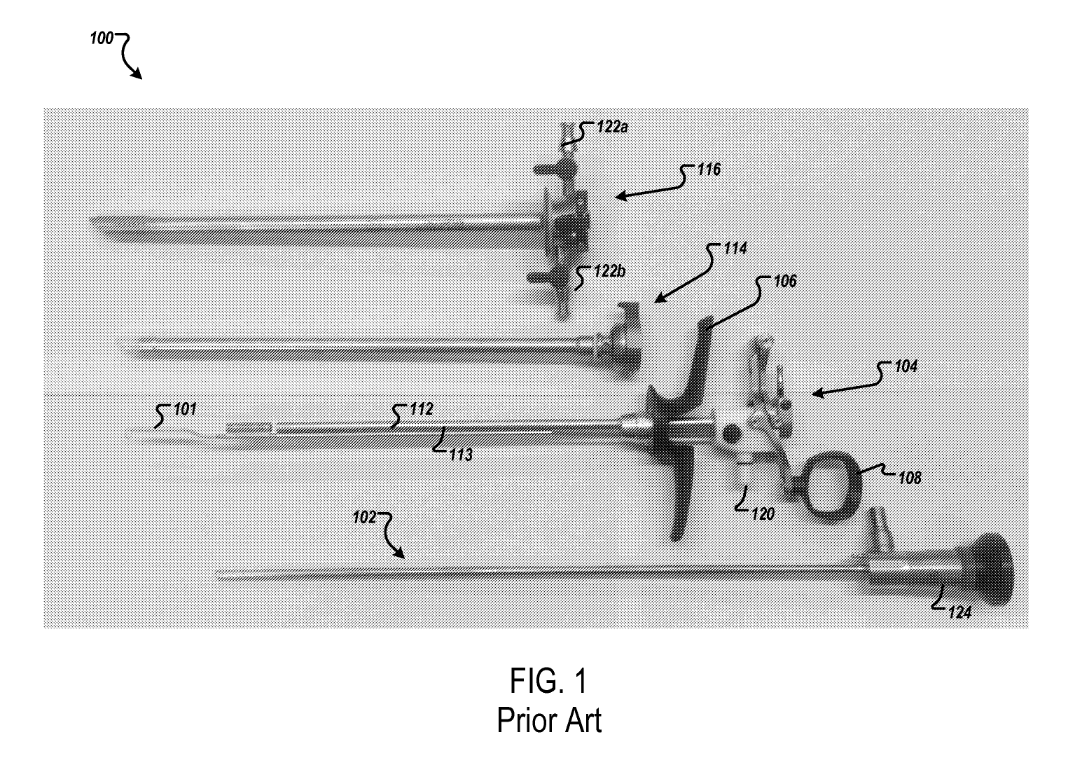

Prior art FIG. 1 shows a typical resectoscope 100 in a disassembled state. The resectoscope 100 includes a telescope 102, a working element 104 including a handle 106, a thumb rest 108, and a cutting element or tool 101, an inner sheath 114, and an outer sheath 116 including an irrigation port 122a-b. The telescope 102 is inserted through the working element 104 and a telescope tube 112. The telescope 102 includes a light port 124 for connection with an optical light source. The cutting element 101 is inserted through the working element and the electrode support tube 113. The inner sheath 114 attaches to the resectoscope 100 over the telescope tube 112, and the outer sheath 116 attaches to the resectoscope 100 over the inner sheath 114. The working element, the sheath assembly, the cutting element, and the telescope are rotationally fixed relative to each other.

Endoscopic surgery is routinely performed to diagnose and treat pathologies affecting areas around natural body openings. For example, a resectoscope can be used during a TURP, a TURBT, an endometrial and fibroid resection, a hysteroscopic surgery, and a resection of polyps and tumors in the rectum during endoscopic gastrointestinal surgery. A resectoscope includes a trigger mechanism to produce a controlled linear movement of the cutting element along the longitudinal axis of the device. As a user engages the trigger mechanism, the cutting element 101, e.g., an electrode tip and/or sharp surface, extends from the outer sheath 116 to the targeted tissue surface. The cutting element is heated and is brought into contact with the targeted tissue. As the user releases the trigger mechanism, the cutting element 101 retracts into the outer sheath 116 and the targeted tissue is severed. In some embodiments, the surgical tool, e.g., cutting element, 101 can have an arcuate shape, e.g., a looped shape. Tissue removal is achieved by reciprocation of the cutting element 101, and thus the cutting element is reoriented to each new tissue section, e.g., to each new lobe. Given the fixed configuration of the cutting element relative to the handle, the user rotates the resectoscope handle to cause reorientation of the surgical tool. In many cases, patient anatomy and/or limited working space require up to and including a 360° rotation to bring the cutting element 101 into contact with all desired surfaces of the targeted area, e.g., the walls and/or lobes of the prostate or bladder. Because the user's hand is incapable of accomplishing a 360° rotation while grasping the handle of this prior art device, the user must release the handle of the device to rearrange their grasp and/or switch the handle of the device to a non-dominant hand to complete the rotation. This leads to a less effective, awkward, and less precise resection.

In contrast to the prior art devices, the present invention provides devices and methods featuring a working element of a resectoscope or other surgical device having a handle that selectively rotates relative to a surgical tool, e.g., a cutting tool. Specifically, the present devices and methods describe that the handle can rotate with the cutting tool as a rigid body (as generally described herein) until the engagement or clutch mechanism is disengaged whereby the rotation of the handle is rendered independent of the position of the surgical tool, e.g., cutting tool. The rotation of the handle continues to remain independent of the cutting tool until the engagement or clutch mechanism is re-engaged. This can be advantageous in situations where the targeted area for tissue removal, e.g., the walls of the prostate or bladder, require any rotation of the cutting tool because this allows the user to selectively maintain an ergonomic hand position, e.g., including a relative hand and/or handle rotation of between 0° and 90°, without forcing the user to reposition or switch hands on the handle, e.g., in cases where the relative handle and/or hand rotation includes between 180° and 270°. Furthermore, these hand positions and/or rotation ranges are self-selected by the user and can, therefore, be dynamically optimized and customized for each user and/or procedure.

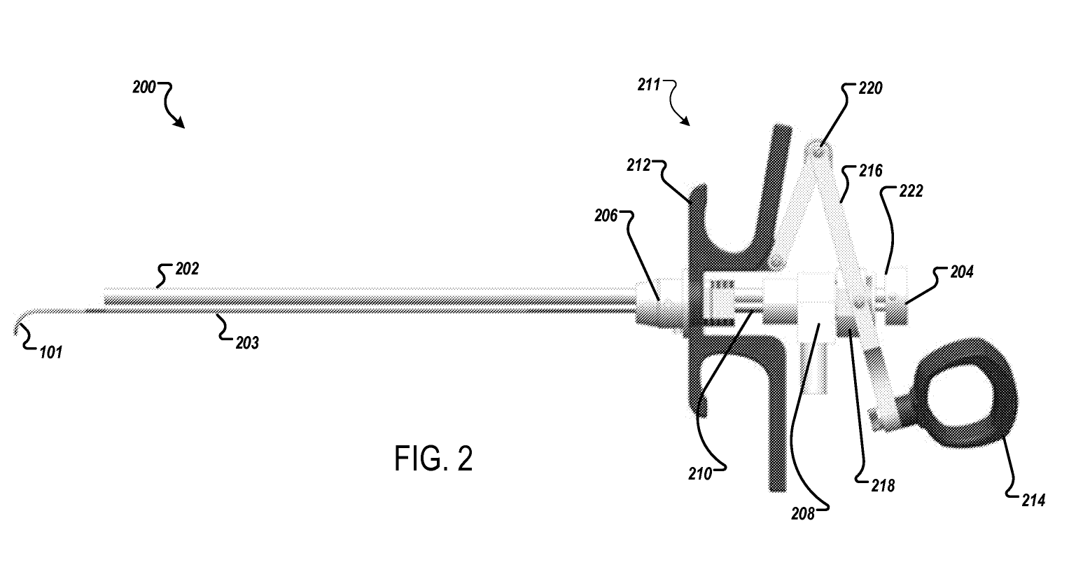

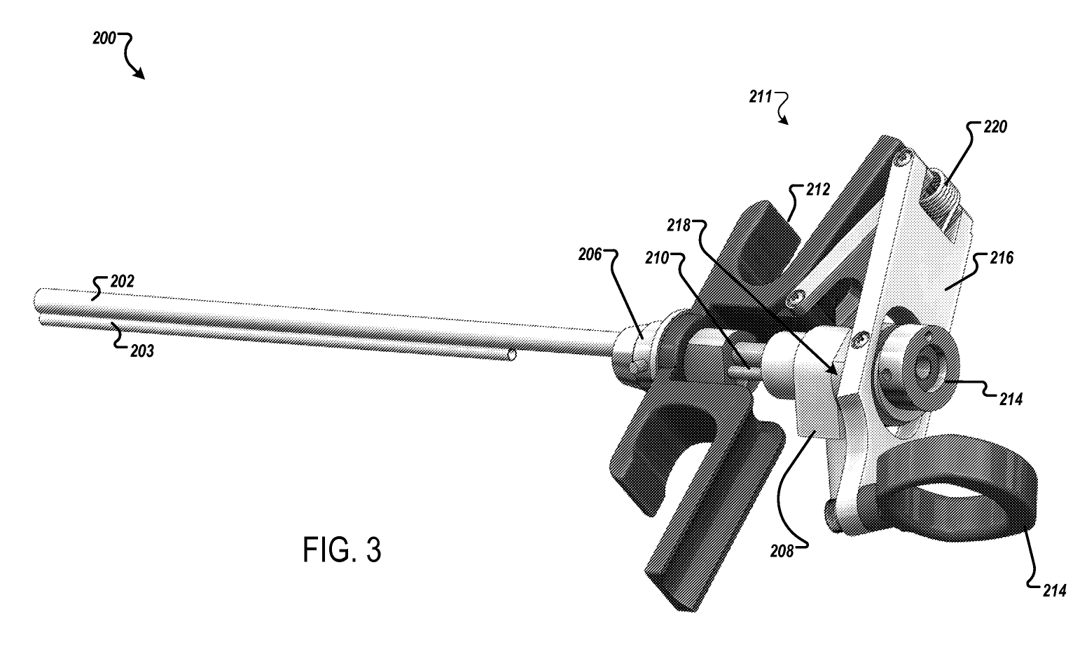

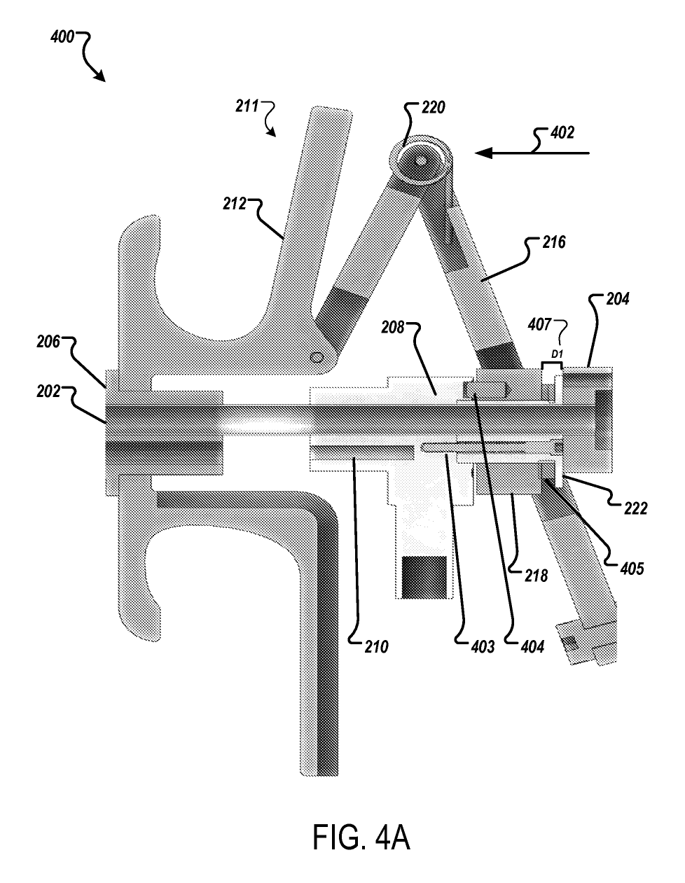

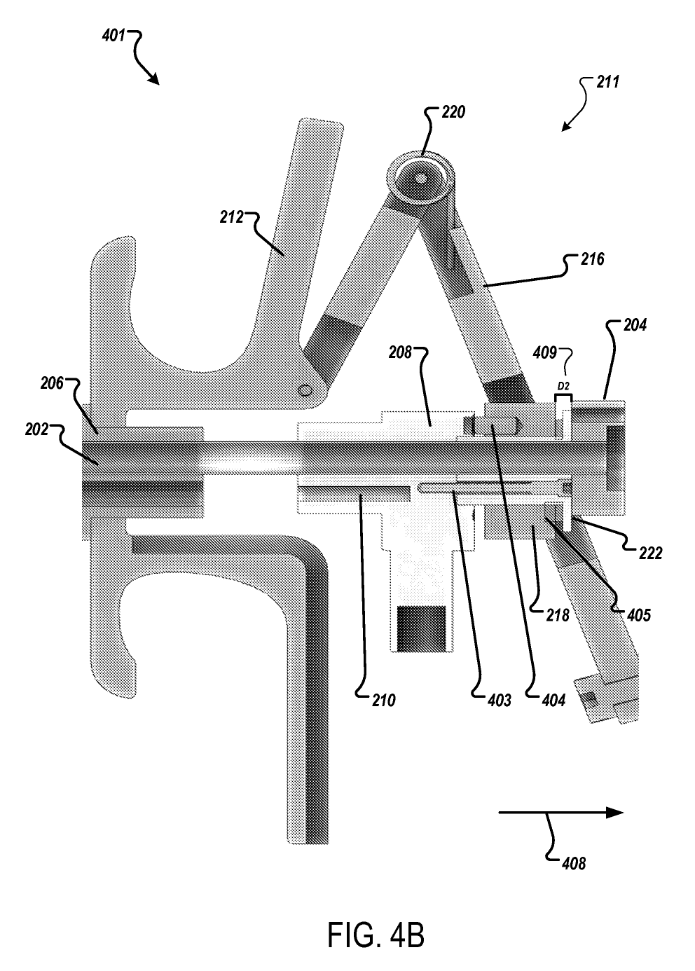

FIGS. 2 and 3 illustrate a working element 200 of a resectoscope configured for compatibility with a standard resectoscope 100 such that assembly of the resectoscope 100 with the new working element 200 described herein rather than prior art working element 104 is indistinguishable to the user. The new working element 200 includes a scope tube 202, an electrode support tube 203 coupled to a front block 206, an electrode block 208, a linear guide rod 210, a rear electrode block 222, and a rear block 204. A handle subassembly 211, including a handle 212, a coupling linkage 216, a torsional spring 220, a clutch mechanism 218, a wave spring 405 (as shown in FIGS. 4A-4B), and a thumb ring 214 is attached to the coupling linkage 216, which is attached to the scope tube 202 between the front block 206 and the rear block 204. The relative rotational movement of the handle subassembly 211 to the scope tube 202 (and thus the cutting element 101) is selectable during use.

The rear block 204 and the front block 206 are linearly and rotationally fixed to the scope tube 202. The electrode block 208 is configured to reciprocate along the scope tube 202 and linear guide rod 210 between the front block 206 and the rear block 204. As shown in FIGS. 2 and 3, the electrode block 208 rests in a neutral position towards the rear block 204. Relative linear movement of the electrode block 208 between the front block 206 and the rear block 204 is effected by either compressing, e.g., squeezing, the thumb ring 214 towards the handle 212 from the neutral position or retracting, e.g., pulling, the thumb ring 214 away from the handle 212. In an assembled device, a forward linear movement along a direction generally designated by an arrow 402 (as shown in FIG. 4A) can cause the cutting element 101 to extend from the resectoscope to provide an exposed cutting surface for tissue removal, while a backward linear movement (arrow 408 shown in FIG. 4B) can cause the cutting element 101 to withdraw and return to a lumen of the resectoscope 100.

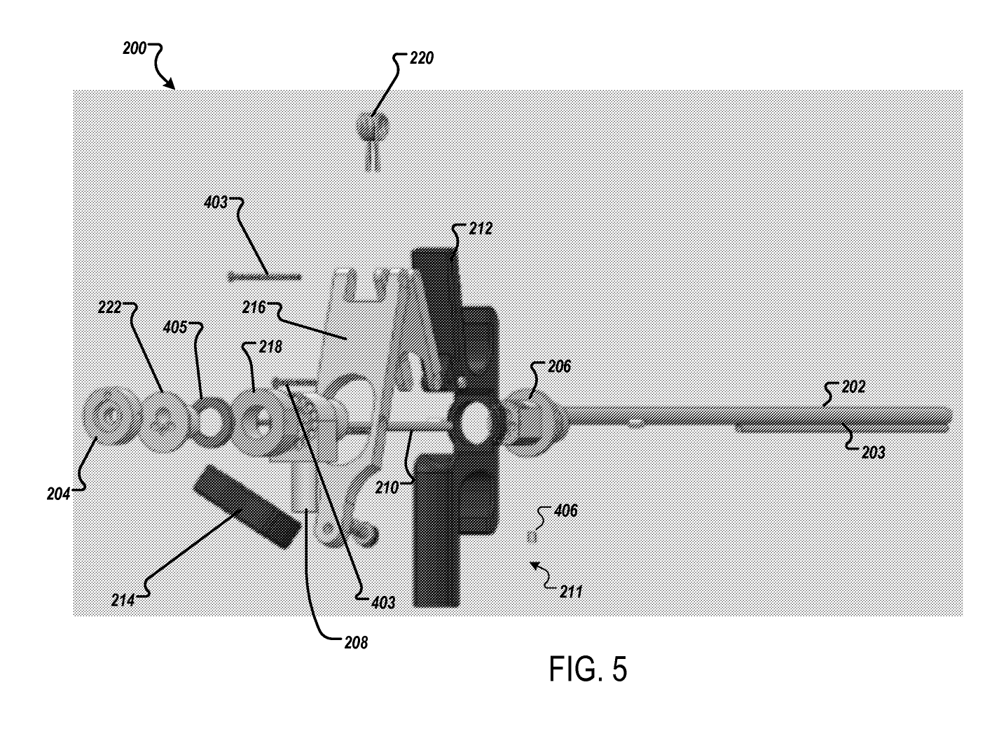

As shown in FIGS. 4A, 4B, and 5, the electrode block 208, including the rear electrode block 222 is rotationally fixed relative to the scope tube 202 and the linear guide rod 210. In a neutral position 400, the handle subassembly 211 is arranged as generally shown in FIGS. 1 and 2. For example, to deploy or extend the cutting element 101 the electrode block 208 is moved in a forward linear direction that is generally represented by the arrow 402. Any linear reciprocation of the electrode block 208 is caused, at least in part, by a movement of the clutch mechanism 218 that is fixed and attached to the coupling linkage 216 of the handle subassembly 211. As shown, the electrode block 208 and the scope tube 202 extend through an opening in the clutch mechanism 218. The handle 212 of the handle assembly 211 is linearly fixed relative to the working element 200 and also fixed rotationally relative to the working element so long as the clutch mechanism 218 is engaged with the (rotationally fixed) electrode block 208.

The clutch mechanism 218 is releasably engaged with the electrode block 208 such that the clutch mechanism can move with the electrode block 208 while the clutch mechanism 218 is engaged. For example, the electrode block 208 also extends through the opening in the clutch mechanism 218 to the rear electrode block 222 to form a flange configuration (as shown in the exploded view of FIG. 5). Screws 403 connect electrode block 208 to rear electrode block 222, e.g., as shown in FIGS. 4A and 4B.

The coaxial (along the longitudinal axis of the device) arrangement of the electrode block 208, the clutch mechanism 218, and the rear electrode block 222 limits the relative linear movement between the clutch mechanism 218 and the electrode block 208. The relative linear movement is also limited by the wave spring 405 which exerts a force on the rear electrode block 222, which causes the clutch mechanism 218 to press against the electrode block 208. For example, the wave spring 405 is selected to exert a force on the clutch mechanism 218 to ensure that the clutch mechanism 218 is rotationally fixed in the neutral position 400. For example, the relative rotational movement between the clutch mechanism 218 and the electrode block 208 is limited (or prevented) due to one or more engagement members 404, e.g., a dowel pin, extending between the clutch mechanism 218 and the electrode block 208. For example, as shown in FIGS. 4A and 4B, the engagement member 404 extends from the clutch mechanism 218 into a corresponding cavity in the electrode block 208. However, the clutch mechanism 218 can be disengaged by a user activation as described below.

In the neutral position 400 the torsional spring 220 within the coupling linkage 216 exerts a force that restrains the clutch mechanism 218 in the neutral position (as shown in FIG. 4A). During use, the user exerts a force on the handle subassembly 211 to overcome this force and thereby causes the clutch mechanism to move. Upon the removal of that force, the torsional spring force returns the handle subassembly 211 to the neutral position 400. For example, in the neutral position 400, the rear electrode block 222 is in contact with and/or pressed against the rear block 204 and the clutch mechanism 218 is positioned away from the rear block 204. In some cases, an engaged distance 407 (D1) between the clutch mechanism 218 and the rear block 204 (as shown in FIG. 4A) is at least as great as the distance that the engagement member 404 extends into the electrode block 208.

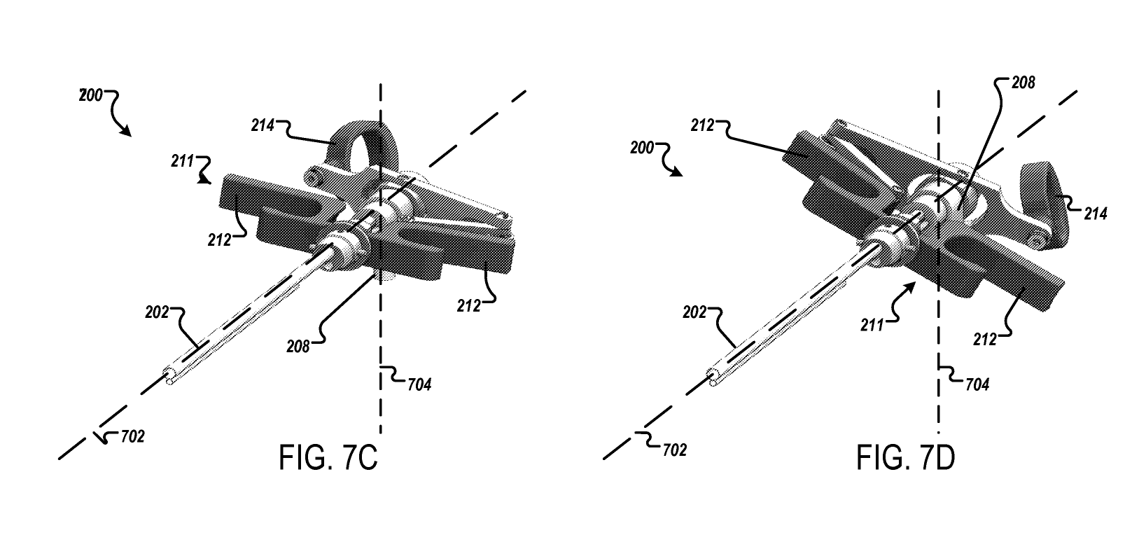

In some cases, the user disengages the clutch mechanism 218 from the electrode block 208 by exerting a force on the subassembly to compress the wave spring 405 against the rear electrode block 222 which is pressed against the rear block 204. For example, in the neutral position 400, the clutch mechanism 218 is separated from the rear block 204 by the engaged distance 407 (D1) between the rear block 204 and the clutch mechanism 218. In some cases, the clutch mechanism 218 remains engaged so long as at least a portion of the engagement member 404 is within the cavity of the electrode block 208. As the user retracts the clutch mechanism (via the handle subassembly 211) along a direction generally shown by an arrow 408, the wave spring 405 compresses, thereby causing a change in distance between the rear block 204 and the clutch mechanism 218, e.g., to a disengaged length 409 (D2) (as shown in FIG. 4B). Once the clutch mechanism 218 is disengaged, the clutch mechanism 218 (and thus the handle subassembly 211) is free to rotate around the scope tube 202 or a longitudinal axis 702 of the working element 200 (as shown in FIGS. 7A-D).

The clutch mechanism 218 is also movable relative to the electrode block 208. For example, the clutch mechanism 218 is removably coupled to the electrode block 208. In a neutral position 400, the clutch mechanism is engaged with the electrode block 208 via an engagement member, e.g., dowel pin, 404. In this example, the clutch mechanism is separated from the rear block by a distance generally represented by D1.

In a disengaged state 401, the wave spring 405 is compressed as the clutch mechanism 218 is retracted towards the rear block 204. In a disengaged state 401, the clutch mechanism 218 is separated from the rear block 204 by a distance generally shown as D2. This retraction causes the dowel pin to retract from the electrode block 208. As such, the clutch mechanism 218 is rotationally free relative to the electrode block 208.

FIG. 5 illustrates an exploded view of the working element 200. As shown, the scope tube 202, including the electrode support tube 203 are attached to the front block 206. The front block 206 extends through an opening in the handle 212, and the linear guide rod 210 extends through the same opening to connect to the front block 206. The linear guide rod 210 also extends into the electrode block 208, which includes a series of holes oriented around the electrode block face. The clutch mechanism 218 is aligned with a wave spring 405 and positioned between the electrode block 208 and the rear electrode block 222. The rear block 204 is joined to the scope tube 202 that extends through the aligned openings in the rear electrode block 222, the spring washer 405, the clutch mechanism 218, the electrode block 208, the handle assembly 211, and the front block 206.

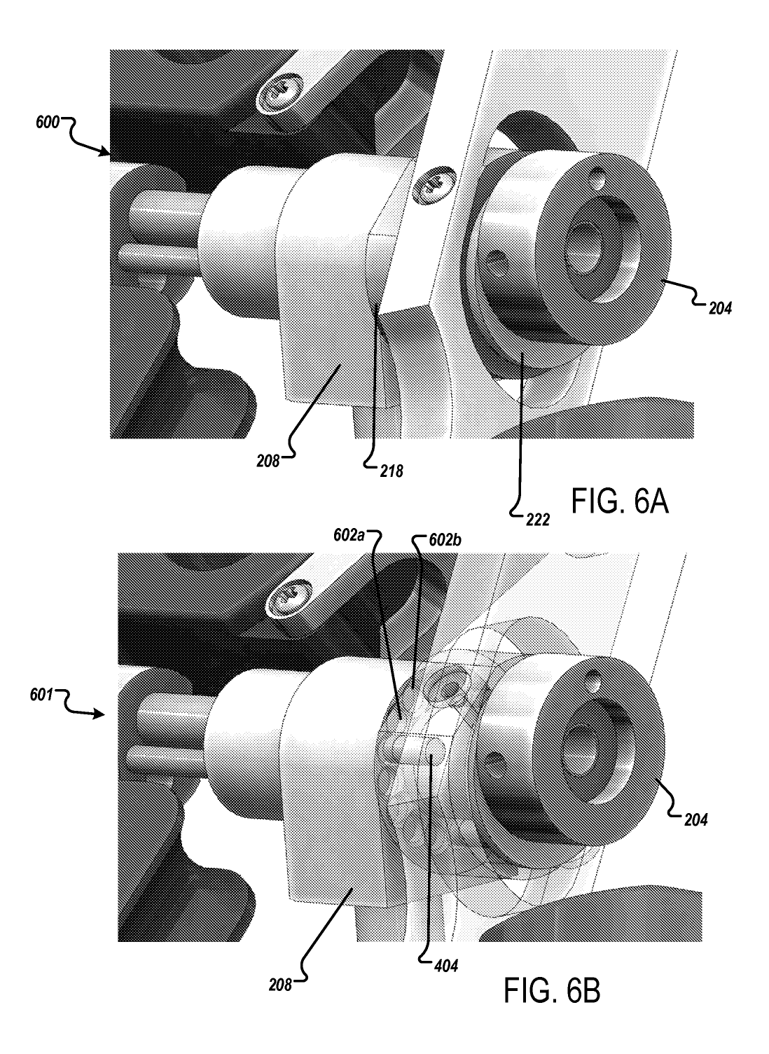

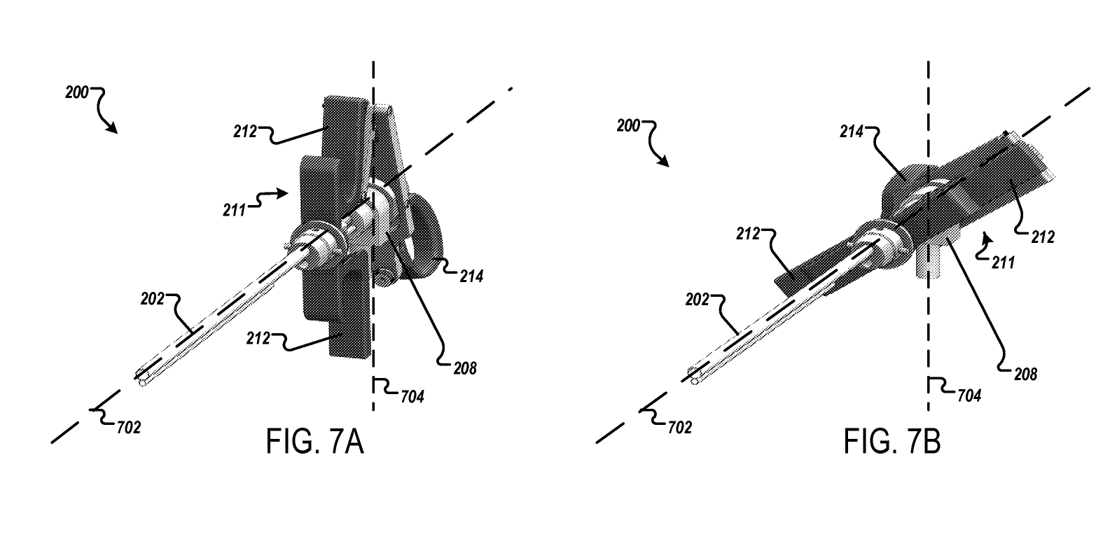

Referring to FIGS. 6A and 6B, an enlarged view 600 of the electrode block 208, the clutch mechanism 218, the rear electrode block 222, and the rear block 204 is shown. As more clearly shown in the partial cutaway view in FIG. 6B, the electrode block includes a plurality of cavities 602a-b, e.g., holes. The engagement member 404 extends from the clutch mechanism 218 into one of the plurality of holes 602a-b. These cavities are arranged in a circular configuration aligned with the rotational path of the engagement member 404, such that the engagement member 404 can align with a cavity 602a-b in a variety of orientations. While a single engagement member 404 is visible, a plurality of engagement members can be used, e.g., two or more, three or more, 4 or more, 5 or more, 6 or more, 7 or more, 10 or more, 12, or more, 14 or more, 16, or more, and so forth. In some examples, the number of the engagement members 404 corresponds to a number of the cavities 602a-b, but there are never more engagement members than there are cavities.

Referring to FIGS. 7A-7D, once the handle assembly 211 is disengaged from the electrode block 208, the working element 200 includes the handle subassembly 211 that rotates relative to a longitudinal axis 702 of the working element. As shown, the handle 212 changes position along a perpendicular vertical axis 704, however, the scope tube 202 and the electrode block 208 remain fixed throughout any rotation of the handle subassembly 211. While not shown here, an optical system connected to the scope tube 202 can also remain rotationally fixed.

The new modified resectoscope including the working element 200 may be used in a number of procedures that include tissue removal such as during a TURP, during a TURBT, during endometrial and fibroid resection during hysteroscopic surgery, and during a resection of polyps and tumors in the rectum during endoscopic gastrointestinal surgery.

In one example, a patient is prepared for a TURP according to known procedures. Once the patient is prepared, the surgeon inserts the modified resectoscope including the working element 200 through the patient's urethra to view and/or access the patient's prostate. As the resectoscope approaches the first tissue portion for resection, the surgeon positions the modified resectoscope such that a surgical tool, e.g., the cutting element 101, would be in proximity to the tissue after it is extended from the resectoscope. In some cases, the surgeon can rotate the resectoscope, and thus the cutting element 101, according to known methods. In some cases, the surgeon can rotate the resectoscope according to known methods until selectively disengaging the clutch mechanism 218 by retracting the thumb rest 214 beyond the neutral position. Once disengaged, the handle subassembly 211 freely rotates relative to the cutting element 101. As such, the surgeon's hand position can be adjusted or reoriented to a more comfortable position. After the surgeon reorients the handle subassembly 211, the surgeon can release the thumb rest back to a neutral position and resume rotating the cutting element 101 according to known methods. After the cutting element 101 is positioned, the surgeon can trigger an extension and a withdrawal of the cutting element 101 to sever the tissue by pressing the thumb rest forward and pulling the thumb rest backwards (or simply allowing the torsional spring to move the thumb rest back) to sever the tissue. As is typical, irrigation can occur during such a resection to clear blood and particulate from the area. This process can be repeated until the target tissue is removed without requiring a surgeon to release the modified resectoscope.

### OTHER EMBODIMENTS

While certain embodiments have been described herein, other embodiments are also possible. For example, the principles of the invention are not restricted to resectoscopes, but are equally applicable to endoscopic and laparoscopic tools requiring rotational movement. In particular, the principles of the invention can be applied to cystoscopes (bladder), bronchoscopes (lungs), and colonoscopes (colon).

The technology described herein is directly applicable to—and may be advantageous to—all manner of laparoscopic or minimally invasive surgery, extending to the general surgical, gynecologic, obstetric, neurosurgical, endoscopic including gastrointestinal, airway intubation with video or without video assistance, and ear, nose, and throat (ENT) fields. A clutching mechanism as described herein stands to be beneficial for similar ergonomic and safety-related reasons in all of the fields mentioned as well as any new or emerging field of procedure that employs an instrument meant to extend and/or resect, treat, and/or manipulate or apply a treatment within the full spectrum of instrument rotation, including up to 360-degrees of rotation or beyond, particularly when the use of a light source is concurrently required.

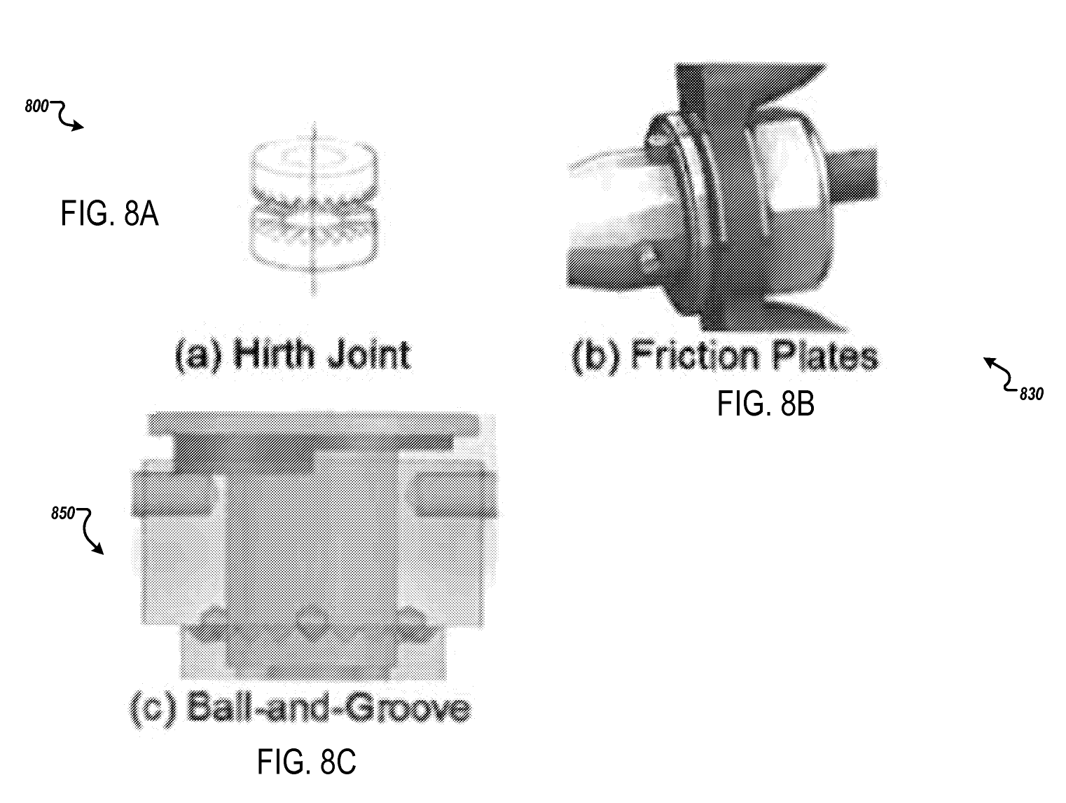

While the use of a pin-and-hole clutch mechanism has been described, other types of clutch mechanisms can be used to selectively engage and disengage a coupling between the handle and the working element. For example, the pin-and-hole mechanism can be replaced by tapered teeth that mesh together on the end faces of two parts, e.g., shafts, similar to a Hirth joint (as shown in FIG. 8A), by friction plates (as shown in FIG. 8B), and/or ball(s) and grooves, e.g., v-grooves (as shown in FIG. 8C). A magnetic clutch can also be used as a clutch mechanism as described herein.

Similarly, while the embodiments of the invention shown in the drawings and described herein utilize a mechanism that disengages the clutch using a mechanism that is activated by the surgeon's thumb other embodiments can be activated using another part of the surgeon's body or by a remote device, e.g., an electronic device, that can be controlled by another person or by the surgeon. In some embodiments a trigger that is activated by the surgeon's index or middle finger may be used. In another embodiment, the surgeon's free hand may toggle a switch, button, or knob to disengage the clutch. In other embodiments, a pedal, button, or switch may be activated by the surgeon's foot. In other embodiments, the clutch may be disengaged electronically.

It is to be understood that while the invention has been described in conjunction with the detailed description thereof, the foregoing description is intended to illustrate and not limit the scope of the invention. It will be understood that various modifications may be made without departing from the spirit and scope of the invention. Accordingly, other embodiments are within the scope of the following claims.

1. A surgical device comprising:
(a) a conduit having a longitudinal axis and being configured to extend into a body lumen; and
(b) a handle coupled to the conduit and configured to selectively rotate relative to the conduit, wherein the handle is coupled to the conduit by a connection comprising a clutch mechanism,
wherein the handle is configured to rotate in response to a user activation of the clutch mechanism.

2. The surgical device of claim 1, wherein the user activation comprises retracting the handle.

3. The surgical device of claim 1, wherein the clutch mechanism comprises one or more members aligned with one or more holes.

4. The surgical device of claim 3, wherein the one or more members are fixed to the handle.

5. The surgical device of claim 1, wherein the clutch mechanism is configured to have an engaged state and a disengaged state, wherein the engaged state rotationally fixes the handle to the conduit.

6. The surgical device of claim 1, further comprising a surgical tool attached to the conduit and aligned parallel to the longitudinal axis of the conduit.

7. The surgical device of claim 6, wherein the surgical tool is rotationally fixed relative to the conduit.

8. The surgical device of claim 6, wherein the surgical tool is a cutting element.

9. A resectoscope comprising:
(a) a conduit having a longitudinal axis and extending through an opening in a handle coupled to the conduit by a connection comprising a clutch mechanism, wherein the handle is configured to selectively rotate relative to the conduit in response to a user activation of the clutch mechanism;
(b) a surgical tool attached to the conduit and rotationally fixed relative to the conduit;
(c) an inner sheath releasably attached to the conduit, wherein the inner sheath surrounds at least a portion of the conduit and the surgical tool; and
(d) an outer sheath releasably attached to the inner sheath, wherein the outer sheath surrounds at least a portion of the inner sheath.

10. The resectoscope of claim 9, wherein the user activation comprises retracting the handle.

11. The resectoscope of claim 9, wherein the clutch mechanism comprises one or more members aligned with one or more holes.

12. The resectoscope claim 11, wherein the one or more members are fixed to the handle.

13. The resectoscope of claim 9, wherein the clutch mechanism is configured to have an engaged state and a disengaged state, wherein the engaged state rotationally fixes the handle to the conduit.

14. The resectoscope of claim 9, wherein the surgical tool is a cutting element.

15. The resectoscope of claim 9, wherein the surgical tool is configured to selectively extend from the outer sheath.

16. A method for examination or treatment within a body cavity or lumen of a patient with a surgical device,
the method comprising:
(1) inserting the surgical device into a body cavity or lumen of the patient such that the surgical device extends into the body cavity, wherein the surgical device comprises
(a) a conduit having a longitudinal axis and extending through an opening in a handle coupled to the conduit, wherein the handle is configured to selectively rotate relative to the conduit and wherein the handle is coupled to the conduit by a connection comprising a clutch mechanism; and
(b) a surgical tool rotationally fixed to the conduit and configured to reciprocate parallel to the surgical device;
(2) rotating the conduit to position the surgical tool within the body cavity; and
(3) selectively rotating the handle about the conduit in response to a user activation of the clutch mechanism, wherein the handle rotates relative to the conduit and the surgical tool.

17. The method of claim 16, wherein the user activation comprises retracting the handle.

18. The method of claim 16, wherein the clutch mechanism comprises one or more members aligned with one or more holes.

19. The method of claim 18, wherein the one or more members are fixed to the handle.

20. The method of claim 16, wherein the clutch mechanism is configured to have an engaged state and a disengaged state and wherein the engaged state rotationally fixes the handle to the conduit.
